(19) United States
(12) Patent Application Publication
(10) Pub. No.: US 2011/0060300 A1
(43) Pub. Date: Mar. 10, 2011

(54) ASPIRATION SYSTEM FOR REMOVING LIQUID DISCHARGED BY THE BODY, AND LIQUID SENSOR THEREFOR

(75) Inventors: Bret Weig, Browns Mill, NJ (US); Mingliang Lawrence Tsai, Holmdel, NJ (US)

(73) Assignee: Conva Tec Technologies Inc., Las Vegas, NV (US)

(21) Appl. No.: 12/673,882

(22) PCT Filed: Aug. 18, 2008
(86) PCT No.: PCT/US08/73494

§ 371 Date: Feb. 17, 2010

Related U.S. Application Data
(60) Provisional application No. 60/956,460, filed on Aug. 17, 2007.

(51) Int. Cl. A61M 1/00

(52) U.S. Cl. 604/319

(57) ABSTRACT

An aspiration system is disclosed for removing body liquid (e.g. urine or a secretion) discharged by the human body. The aspiration system comprises a body interface device with a liquid sensor, and an aspiration unit coupled to the body interface device. The liquid sensor comprises a temperature sensor and/or a resistance bridge. The aspiration unit includes: a vacuum chamber; a pump for pre-charging the vacuum chamber with a vacuum; a valve coupled between the vacuum chamber and the body interface device; and a control circuit for controlling the valve to apply aspiration suction from the vacuum chamber to the body interface device in response to detection of body liquid at the liquid sensor.

### FIELD OF THE INVENTION

The present invention relates to an aspiration system for removing liquids, discharged by the human body. The invention is especially suitable for removing urine, but the invention is not limited only to urine, and may be used for other body fluids and secretions.

### BACKGROUND TO THE INVENTION

U.S. Pat. Nos. 5,002,541, 4,747,166 and 4,631061 describe human urine aspiration systems. The urine removal systems include an electric pump for applying suction to a urinal. The urinal includes a liquid sensor for automatically activating the pump when the presence of liquid urine is detected. The three patents all describe liquid sensors in the form of electrodes forming a normally open circuit that is closed by conduction through liquid, when the sensor is contacted by liquid. The last patent also describes an alternative liquid sensor in the form of an optical sensor.

The use of such an automatic aspiration system for removing urine has the potential to provide significant advantages compared to conventional urine management techniques. For example, the system can avoid the need for a patient to wear diapers to collect urine, with the consequent burden on hospital staff or other caregivers to frequently check and change the diaper. The system can also avoid the need for an in-dwelling urinary catheter for draining urine. There is a significant rate of nosocomial urinary tract infections contracted by patients who are catheterized for substantial lengths of time. Such infections are very serious because they can be fatal and, at the very least, lead to increased recovery times in the hospital and additional cost and burden for hospital staff and caregivers.

It would be desirable to improve on prior art designs of aspirated liquid removal systems, in order to satisfy commercial need and improve customer acceptance. In devising the present invention, the inventors appreciated that one aspect that would benefit improvement is in the speed of response of the system to aspirate urine. The present invention has been devised bearing these issues in mind.

### SUMMARY OF THE INVENTION

In devising the present invention, it has been appreciated that it would be desirable to improve the liquid sensing techniques of the prior art. Previous liquid sensing techniques may be quite insensitive, by having to rely on a sufficient quantity of liquid to effectively short circuit two electrodes, or by having to provide electrodes over a large area of a urinal to provide sufficient conduction sensitivity.

In one aspect, the invention provides a liquid sensor for an aspiration system for removing body liquids discharged by the human body, the liquid sensor being a temperature sensor.

The use of a temperature sensor can provide a reliable and quick indication of body liquid exiting the body, without requiring the sensor to be dispersed over a large area. Liquid, such as urine, exits the body at a temperature of about 37° C., which is notably higher than ambient room temperature (typically about 23° C.), and also higher than the ambient temperature of a sensor positioned close to the human skin (typically about 32° C.).

The presence of liquid is detected by detecting (i) a rapid change in temperature and/or (ii) a temperature rise above a threshold, such as above 36° C.

In another aspect, the invention provides a liquid sensor for an aspiration system for removing body liquids discharged by the human body, the liquid sensor comprising a resistance bridge circuit generating a bridge output that is responsive to contact of one of more bridge elements with liquid. The bridge circuit comprises at least one element whose resistance varies when contacted by liquid. The element comprises first and second spaced apart electrodes.

The liquid sensor may further comprise a monitoring circuit for monitoring the bridge output.

The use of a bridge circuit provides a highly sensitive liquid detector, by enabling a moderate change in resistance to be easily detected. This provides a faster response than, for example, relying on a sufficient quantity of liquid to completely short circuit two electrodes.

Also, in devising the present invention in another aspect, the inventors appreciated that it would be desirable to improve the manner of application of aspiration suction once liquid has been detected. The techniques of the above prior art inherently involve delay while the suction pump gets up to speed. It may be possible to employ a more powerful pump, but this would add significantly to the cost, weight and power consumption of the apparatus.

In accordance with this aspect, the invention provides an aspiration system for removing body liquids discharged by the human body, comprising: a vacuum chamber; a pump for charging the vacuum chamber with a vacuum; a body interface device (e.g., urinal) through which liquid is collected and having a liquid sensor; an electronically controlled valve for controlling the application of vacuum from the chamber to the body interface device in order to suck liquid away from the body interface device; and a control circuit for controlling the valve.

With such an arrangement, a vacuum can be applied more quickly from the pre-charged vacuum chamber, than waiting for a pump to start-up, and achieve a normal pumping capacity. This facilitates rapid response and removal of a large quantity of liquid from the body interface device, and thereby reduce the likelihood of spill or saturation of the body interface device. The use of a vacuum chamber also provides a greater instantaneous suction than the volumetric capacity of the pump.

The valve may be a variable aperture valve, for regulating variably the amount of suction according to the aperture size. Alternatively, the valve may be an open/close valve. The control circuit may be configured to regulate the effective aperture of an open/close valve by generating a pulsed control signal. The effective aperture is defined by the ratio of on-time to off-time of the pulsed control signal. Alternatively, the valve may be a change-over valve for selecting between two or more different suction paths from the vacuum chamber, having different resistances. The paths may include a relatively unrestricted path for applying large suction from the vacuum chamber, and a relatively restricted path for applying a smaller suction.

In another general aspect, the invention provides an aspiration system for removing body liquid (e.g., urine or a secretion) discharged by the human body. The aspiration system comprises a body interface device with a liquid sensor, and an aspiration unit coupled to the body interface device. The liquid sensor comprises a temperature sensor or a resistance bridge. The aspiration unit includes: a vacuum chamber; a pump for pre-charging the vacuum chamber with a vacuum; a valve coupled between the vacuum chamber and the body interface device; and a control circuit for controlling the valve to apply aspiration suction from the vacuum chamber to the body interface device in response to detection of body liquid at the liquid sensor.

As used throughout this specification, the term “vacuum” refers to any pressure below ambient atmospheric pressure. In one form, the term “vacuum” refers to a pressure below 101 kPa. The reference to “charging” a vacuum means removing air to generate a vacuum. Also, as used herein the term “urinal” refers to any device of any form or shape configured for receiving urine directly from a human body orifice (such as a cup-shaped female urinal, a male condom urinal, or a urostomy body fitment).

Although features believed to be of significance have been highlighted above and/or in the claims, the Applicants may seek claims protection for any novel feature or idea disclosed herein and/or illustrated in the drawings, whether or not emphasis has been placed thereon.

### BRIEF DESCRIPTION OF THE DRAWINGS

FIG. 1 is a schematic block diagram of a first embodiment of liquid removal system.

FIG. 2 is a schematic cut-away view of the urinal of the liquid removal system.

FIG. 3 is a schematic circuit diagram of a resistance bridge.

FIG. 4 is a schematic block diagram of a second embodiment of liquid removal system.

### DETAILED DESCRIPTION OF PREFERRED EMBODIMENTS

The same reference numerals are used to depict the same or equivalent features in each of the embodiments described below.

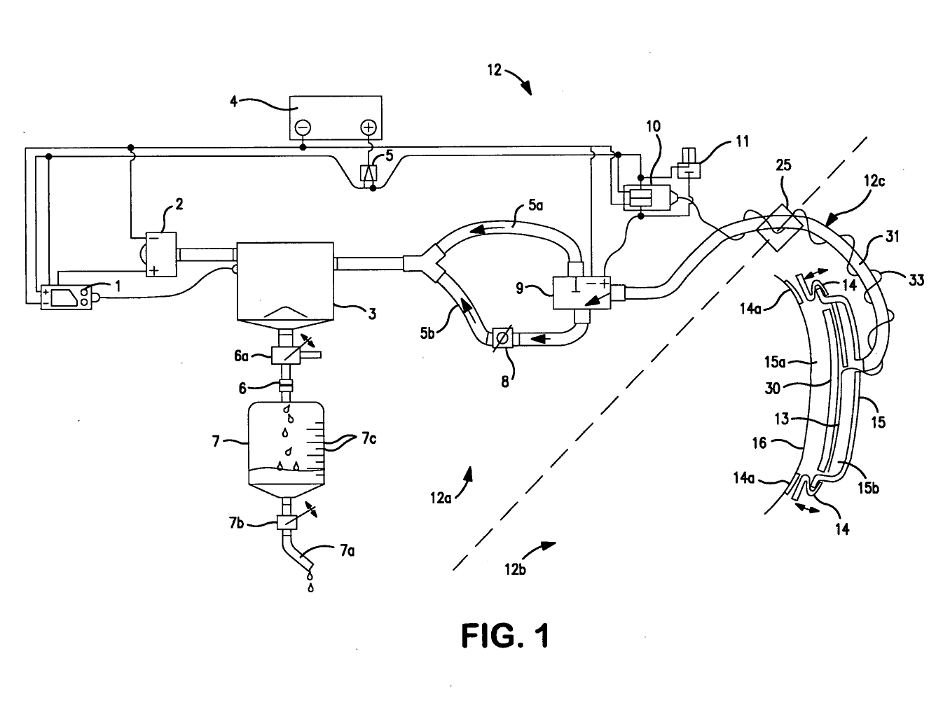

Referring to FIG. 1, an aspiration system 12 for removing body liquid discharged by the human body generally comprises an aspiration unit 12a and a body interface device 12b coupled to the aspiration unit 12a by means of a flexible conduit 12c. The flexible conduit 12c may be regarded as part of the aspiration unit 12a and/or part of the body interface device 12b. The body interface device 12b is configured for fitting to the body at, or around, the site of discharge of the body liquid desired to be collected. In the example of urine removal, the body interface device 12b is configured to fit at the genital region of a male or female wearer, or a surgical urostomy. For females, the body interface device 12b is in the form of a snug-fitting urinal (as illustrated herein). For males, the body interface device 12b is in the form of a condom (not illustrated). For urostomates, the body interface device 12b is in the form of a stoma fitment (not illustrated). The present embodiment is especially suitable for removing urine, since urine removal is most challenging in terms of the volume of the liquid to be removed and the speed of liquid discharge. The speed of response of the apparatus to detect and remove the liquid is a significant factor in the ability of the apparatus to manage a urine discharge.

The aspiration unit 12a comprises a vacuum chamber 3, a pump 2 for removing air from the vacuum chamber 3 to charge the vacuum chamber 3 with a vacuum, an electronically controlled control valve 9 for controlling the application of vacuum from the vacuum chamber 3 to the body interface device 12b, and a control circuit 10 for controlling the valve 9. The pressure within the vacuum chamber 3 is monitored by a pressure sensor switch 1, which in turn controls the application of electrical power from a power supply 4 (via a master device on/off switch 5) to the pump 2. When the pressure is above a predetermined threshold (such as 87.6 kPa), the pressure sensor switch 1 closes to operate the pump 2 in order to pump out air, and charge the vacuum chamber 3 with a vacuum. Once the pressure drops below the threshold, the pressure sensor switch 1 opens to stop the pump 2. The pressure sensor switch 1 continues to monitor the pressure in the vacuum chamber 3, and to operate the pump 2 as necessary to maintain a low pressure vacuum within the vacuum chamber 3. The pressure sensor switch 1 may optionally include hysteresis in the switching threshold to avoid the pump 2 being activated continuously in a short intermittent manner. Additionally or alternatively, the pump 2 may be controlled to continue pumping for predetermined time duration after the switching threshold has been achieved.

In the present embodiment, the control valve 9 is a 3-port solenoid operated changeover valve that couples the body interface device 12b to the vacuum chamber 3 via one of two parallel paths 5a, 5b. Path 5a is a substantially unrestricted high-vacuum path for applying full suction to the body interface device 12b, in order to remove liquid when detected. Path 5b is a relatively restricted, low-vacuum path (more restricted than the high-vacuum path 5a), for applying moderate suction (or no suction) to the body interface device 12b in a quiescent state of the apparatus. In one form, it may be desirable to cut all suction; in another form, it may be desirable to maintain a moderate suction to keep the body interface device dry, to remove small drips of liquid or other contaminants, and/or to cause the body interface device 12b to gently hug the skin. A needle valve 8 in path 5b permits the low-vacuum level to be regulated to a desired amount, or for suction to be cut-off by closing the needle valve 8. In the present embodiment, as a failsafe, the control valve 9 is configured to select the low-vacuum path 5b when no electrical power is applied to the control valve 9 and the solenoid is deactivated, and to select the high vacuum path 5a only when electrical power is applied to the control valve 9 to activate the solenoid. Although only two paths 5a, 5b are illustrated, it will be appreciated that additional parallel paths of different flow resistance may be provided, and between which the control valve 9 selects a path, in order to increase the finesse of control of suction.

The control circuit 10 receives an input from a liquid sensor 13 located in the body interface device 12b. The electrical signal from the liquid sensor 13 is transmitted to the control circuit 10 by means of electrical cable 33. In one form, the liquid sensor 13 comprises a temperature sensor for measuring the temperature at the body interface device 12b. When body liquid such as relatively warm urine or body secretion is discharged into the body interface device 12b, the temperature increases. The presence of body liquid is detected by a temperature sensor sensing a temperature value exceeding a preset threshold value between 30° C. and 42° C. and/or a rate of increase in temperature of at least 1° C. within two seconds. The use of a temperature sensor can provide a reliable and rapid indication of the presence of discharged body liquid.

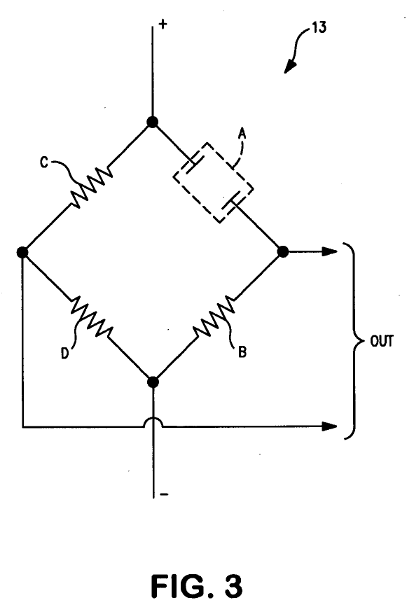

In another form, the liquid sensor 13 comprises a resistance bridge circuit, such as that illustrated in FIG. 3, and comprising at least one element A that is responsive to liquid. The element A may have a resistance responsive to the presence of liquid. The element A may comprise two spaced apart terminals. The use of a resistance bridge circuit can provide a liquid sensor that is sensitive to variation in electrical resistance even in the presence of a small quantity of liquid.

Alternatively, the liquid sensor may be a combination of both of the above, comprising a temperature sensor in a resistance bridge circuit.

In use, the control circuit 10 monitors the output from the liquid sensor 13 to detect presence of body liquid entering the body interface device 12b. When liquid is detected, the control circuit 10 activates the control valve 9 to select the high vacuum path 5a for applying full suction from the vacuum chamber 3 to the body interface device 12b. The liquid is sucked out of the body interface device 12b, via the flexible conduit 12c, the control valve 9 and the high-vacuum path 5a to the vacuum chamber 3. At the vacuum chamber 3, the liquid drops down under gravity into a collection chamber 7. The collection chamber 7 may optionally be accommodated in the aspiration unit 12a, or it may be coupled to an external fixture of the aspiration unit 12a. In either case, the collection chamber 7 may be removably coupled by a suitable connector 6, for cleaning, emptying or replacement. Preferably the connector 6 is a quick disconnect connector. A connector valve 6a prevents escape of vacuum in the vacuum chamber 3 when collection chamber 7 is removed. The connector valve 6a may be operated manually, or automatically when the connector 6 is disconnected. The collection chamber 7 may optionally include a drain output 7a with a drain valve 7b that may be toggled between open and closed conditions. The collection chamber 7 may also optionally be transparent with graduations 7c, or include a graduated window, so that the amount of collected liquid can be visually inspected and measured.

The use of the pre-charged vacuum chamber 3 enables a high vacuum to be applied to the body interface device 12b rapidly, in order to quickly remove even a large quantity of liquid, without any delays associated with a vacuum pump starting or reaching full pump speed. Also, liquid can be sucked away from the body interface device at a greater instantaneous volume capacity than the volumetric capacity of the pump. Instead, the pump 2 is used to pre-charge the vacuum chamber 3 with a vacuum, and to maintain or re-charge the vacuum in the vacuum chamber 3 over time. The pump 2 may therefore be relatively small and/or lightweight as desired.

Once the majority of the liquid has been sucked away, the liquid sensor 13 will generate a non-liquid output signal. For example, in the case of a temperature sensor, the temperature will decrease towards ambient temperature. In the case of a resistance bridge, the resistance will return to its pre-liquid value. In response, the control circuit 10 deactivates the control valve 9 to apply the low vacuum (or no suction, if desired) for quiescent operation. The control circuit 10 may either deactivate the control valve 9 immediately upon the absence of liquid being detected, or after a predetermined time delay subsequent to the absence of liquid being detected.

A manual switch 11 is also provided, coupled to the control unit 10, for manually commanding high vacuum suction from the vacuum chamber 3 when desired. The manual switch 11 provides additional versatility for the patient or caregiver, as well as a back-up failsafe control. The high vacuum suction may be applied either for a preset time (using a timer circuit in the control circuit 10), or while the manual switch 11 is continued to be depressed (in the case of a spring-loaded press-to-make switch) or until the manual switch 11 is toggled or operated again to command a stop of the high vacuum suction.

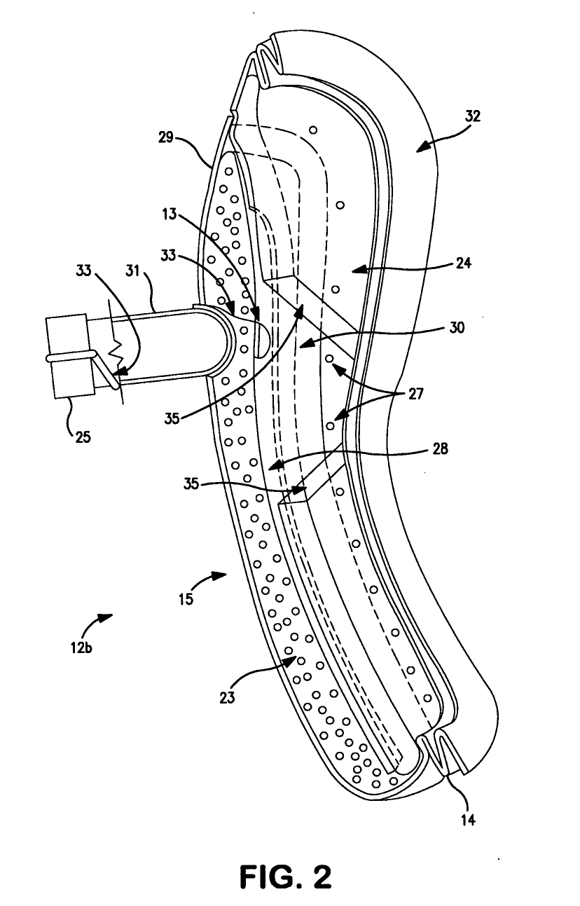

Referring to the general view of FIG. 1, and the more detailed view of FIG. 2, the body interface device 12b is removably attachable and re-attachable to the body to facilitate cleaning. A cover component 15 of the body interface device 12b may have a flexible boot or skirt 14 located near the body attachment 14a for comfort and to provide a seal to the body. The boot 14 may impart a hugging action when vacuum is applied. The body attachment 14a may adhesively engage the skin 16 and the boot 14. The cover component 15 comprises an outer shell made of soft semi-rigid and/or flexible material. Two chambers are configured within the cover 15, and are divided by a chamber divider 30. In a first or inner chamber 15a, at a mouth area where the cover component 15 is most proximal to the body is an inner open cell foam 24 which covers the entire inside area of the cover 15 and is the primary element of the first, inner chamber 15a. A plurality of small air inlets 27 is provided around the periphery and underneath the inner open cell foam 24.

The chamber divider 30 is provided directly underneath the inner open cell foam 24, and is made of a material more resistant to fluid penetration than the inner open cell foam 24. The chamber divider 30 is attached in such a way as to provide a seal to the outer shell 29. The chamber divider 30 has a center slit opening 28 through which the removing fluid will pass. The air inlets 27 are positioned so as to provide an air flow from the inlets 27 across the surface of the chamber divider 30 and into the center slit opening 28, and thereby direct fluid toward the center slit opening 28. As an alternative to a single center slit opening 28, the chamber divider 30 could comprise plural apertures of holes over its surface.

Below the chamber divider 30 is an outer open cell foam 23 which supports and is trapped by the chamber divider 30 and the outer shell 29. The liquid sensor 13 (temperature sensor or resistance bridge) is positioned on top of the outer open cell foam 23, so as to be in the middle of the center slit opening 28 and in direct line with e.g., the urethra (in a female urine removal system). The connecting conduit 31 is attached to the outer shell 29 and/or the outer open cell foam 23, and a hole in the outer shell 29 where the connecting conduit 31 is located is in line with the urethra and liquid sensor 13, and provides the pathway for the liquid to be sucked away. The electrical cable 33 from the liquid sensor 13 passes through the outer open cell foam 23, through the outer shell 29 and accompanies the connecting conduit 31 towards the aspiration unit 12a. The connecting conduit 31 and the electrical cable 33 include a quick disconnect 25 to allow (i) the wearer to temporarily disconnect from the aspiration unit 12a without removing the body interface device 12b from the body, and/or (ii) replacement of the body interface device 12b or its cover component 15. The quick disconnect 25 may comprise separate connectors for the suction connecting conduit 31 and the electrical cable 33, or the quick disconnect 25 may integrate the connectors for both in a common connector housing.

In another embodiment of the body interface device 12b, the quick disconnect 25 may have one portion located near the proximal end of the connecting conduit 31 where the leading end of the connecting conduit 31 is inserted into an opening in the cover component 15 and into the outer chamber 15b. The liquid sensor 13 may be mounted on the outer surface of the connecting conduit 31, and the quick disconnect 25 configured, such that the liquid sensor 13 with the connecting conduit 31 passes through another portion of the quick disconnect 25 specific to the body interface device 12b and aligns with the center slit opening 28 so as to be in line with the urine stream. The quick disconnect 25 has a feature that provides this alignment. The connecting conduit 31 has a plurality of openings near its end such that the openings are within the outer chamber 15b when connected to the body interface device 12b. the open end of the connecting conduit 31 may have a restriction so as to effect the vacuum over the other openings near the end of the connecting conduit 31. Also, the distal end of the connecting conduit 31 and the electrical cable 33 may be able to be disconnected from the aspiration unit 12a (not shown). This embodiment enables the connecting conduit 31 and the liquid sensor 13 to be completely removed from the body interface device 12b and connected to a new body interface device 12b or other interface used in the collection of liquid discharged from the body. Further, the connecting conduit 31 with the liquid sensor 13 may be replaceable, allowing the aspiration unit 12a to be reused with new connecting conduits and sensors.

When urination or liquid secretion occurs, the liquid passes through the inner open cell foam 24 and contacts the liquid sensor 13. The control circuit 10 operates the control valve 9 to apply a high vacuum, as described earlier. The outer chamber consisting of the outer open cell foam 23 is de-pressurized by the applied high vacuum pulling through the center slit opening 28. Atmospheric air is pulled through the air inlets 27 across the top surface of the chamber divider 30, in order to channel expelled liquid through the center slit opening 28 into the outer chamber and connecting conduit 31 and away from the body.

The present embodiment is especially suitable for urine removal from the human body, and the illustrated body interface device 12b is shaped as a urinal for fitting to the female body. However, it will be appreciated that the body interface device 12b could be shaped as a condom for fitting to a male's penis. It will also be appreciated that the urinal body interface device 12b could be configured for connection to a stoma of a urostomate. Other configurations of the body interface device 12b may be provided depending on the type of liquid or secretion to be collected from the body.

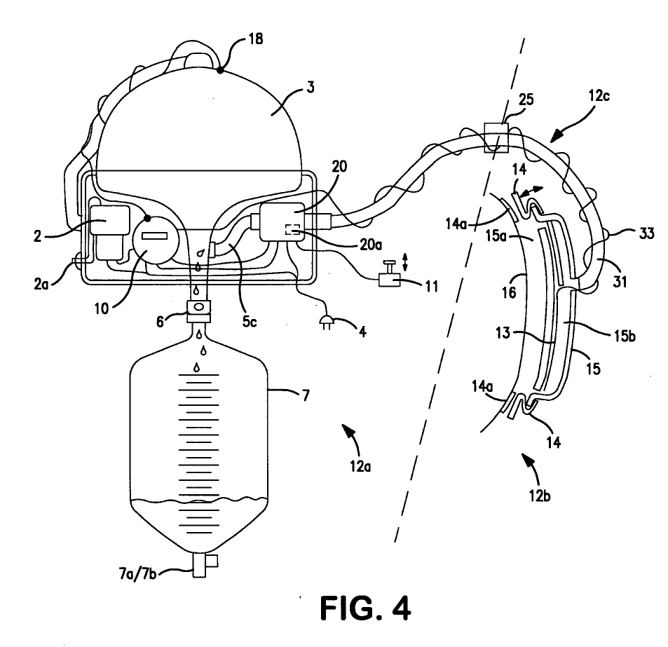

FIG. 4 illustrates a second embodiment of liquid removal system that is similar to the first embodiment. The main difference is that, in the second embodiment a single conduit path 5c is provided between the vacuum chamber 3 and the body interface device 12b. The amount of vacuum applied from the vacuum chamber 3 to the body interface device 12b, is regulated variably by a control valve 20, under the control of the control circuit 10.

The control valve 20 may have a variable aperture or orifice. Variable aperture valves include a continuously variable aperture valve, such as a servo position control valve, or a valve having a plurality of predetermined discrete aperture sizes. In either case, the position of the valve member, and the aperture size, are controlled by the control signal from the control circuit 10.

Alternatively, the control valve 20 may be of an on/off type, and the effective aperture controlled by means of a pulse modulated open/close control signal. The effective aperture depends on the mark:space ratio of the control pulses, which defines the relative on (open):off (closed) durations.

The control circuit 10 is configured to generate a control signal that controls the control valve 20 to regulate variably the vacuum applied to the body interface device 12b. The control circuit 10 receives inputs from the liquid sensor 13, and from a vacuum sensor 18 (which replaces the pressure sensor switch 1 of the first embodiment). The control circuit 10 optionally receives a further input from a fluid flow sensor 20a. The fluid flow sensor 20a measures the flow rate through the conduit 5c. The fluid flow sensor 20a may conveniently be included in the control valve 20. In addition to controlling the control valve 20, the control circuit 10 controls the pump 2. The control circuit 10 may also receive a manual command input from a manual switch 11, as in the first embodiment.

The function of the aspiration system 12a is similar to that described for the first embodiment, except that the control circuit 10 oversees the control of the pump 2, and the control valve 9, depending on the inputs from the vacuum sensor 18, the liquid sensor 13 and the fluid flow sensor 20a. The control circuit 10 controls the control valve 20 to provide high vacuum and low vacuum (or no vacuum) states, by controlling the valve aperture.

The control circuit 10 may operate to determine the amount or rate of liquid entering the body interface device 12b, instead of merely detecting the presence or otherwise, and control the control valve 20 variably to apply a vacuum amount corresponding to the amount of liquid, in order to remove the liquid at a rate based on the rate at which the liquid is discharged from the body. This may be more comfortable for the wearer.

Additionally or alternatively, depending on the rate of fluid flow, the control circuit 10 may be configured to activate the pump 2 earlier than normal, in order to preserve a vacuum in the vacuum chamber 3 for as long as possible.

The control circuit 10 may include a micro-controller, configured to respond to inputs in a pre-programmed manner, such as by using the input signals to access a pre-programmed information map defining the output control signals to be generated. The control circuit 10 may either judge vacuum level switching, and the liquid detection, in the control circuit 10 based on variable sensor inputs, or the judgment may be implemented in the sensors themselves. The switching thresholds of the sensors may be programmable by the controller.

It will be appreciated that the foregoing description is merely illustrative of preferred embodiments of the invention, and that many modifications, improvements and equivalents may be within the scope of the invention as claimed.

We claim:

1. A liquid sensor for use in an aspiration system for removing liquids discharged by the human body, the liquid sensor being configured to sense the presence of discharged body liquid, wherein the liquid sensor a temperature sensor; and/or a liquid responsive resistance bridge.

2. The liquid sensor of claim 1, wherein the liquid sensor is the temperature sensor, and further comprising a control circuit for generating an output signal indicating the presence of body liquid, when the temperature is detected to exceed a predetermined threshold of between 30° C. to 42° C.

3. The liquid sensor of claim 1, wherein the liquid sensor is the temperature sensor, and further comprising a control circuit for generating an output signal indicating the presence of body liquid, when the temperature is detected to exceed a predetermined rate of increase.

4. The liquid sensor of claim 2, wherein the threshold is between 30° C. to 42° C.

5. The liquid sensor of claim 2, wherein the threshold is an increase in temperature of at least 1° C. within two seconds.

6. The liquid sensor of claim 1, wherein the liquid sensor is the liquid responsive resistance bridge, and wherein the resistance bridge comprises at least one element having a resistance responsive to contact by liquid.

7. The liquid sensor of claim 1, further comprising a control circuit configured to generate an output signal indicating the presence of body liquid, when a change is sensed that exceeds a change threshold.

8. A body interface device for fitting to the human body as part of an aspiration system for removing liquids that are discharged into the body interface device by the human body, the body interface device comprising:
a housing accommodating a suction passage; and
a liquid sensor for detecting the presence of discharge body liquids in the body interface device, wherein the liquid sensor is a temperature sensor;
and/or a liquid responsive resistance bridge.

9. The body interface device of claim 8, further comprising an aspiration connector for releasably connecting the suction passage to an aspiration passage of an aspiration unit, and an electrical signal connector for connecting the liquid sensor electrically to the aspiration unit.

10. The body interface device of claim 8, wherein the housing accommodates first and second chambers separated by a chamber divider, wherein:
the first chamber is provided at an interface mouth of the housing to provide an offset from the body and a volume for initial release of body liquid;
the second chamber is provided behind the first chamber for application of suction to the first chamber.

11. The body interface device of claim 9, wherein the aspiration passage and electrical signal connector are releasably connected to the aspiration unit, and the liquid sensor is attached to the aspiration passage.

12. The body interface device of claim 10, wherein at least one of the first and second chambers contains an open cell foam material, and the chamber divider is configured to be less permeable than the open cell foam material.

13. The body interface device of claim 10, wherein the chamber divider is configured to channel fluid flow between the chambers.

14. The body interface device of claim 10, wherein the chamber divider comprises a wall of generally liquid impermeable material, having one or more gaps therein.

15. The body interface device of claim 14, wherein the gap is in the form of a slit.

16. The body interface device of claim 14, wherein the gaps comprise plural apertures.

17. The body interface device of claim 10, wherein the housing comprises a plurality of air inlet apertures communicating with the first chamber, in order to induce an air flow for carrying liquid from the first chamber into the second chamber.

18. The body interface device of claim 10, wherein the liquid sensor is positioned in intimate communication with at least one of the chambers.

19. An aspiration system for removing body liquids discharged from the human body, the system comprising:
a body interface device for fitting to the human body for receiving body liquids discharged by the body, the body interface device comprising a liquid sensor selected from a group consisting of a temperature sensor;
and/or a liquid responsive resistance bridge;
an aspiration suction source; and
a control circuit responsive to the output of the liquid sensor, and configured to control the application of aspiration suction to the body interface device in order to remove the body liquids.

20. An aspiration system for removing body liquids discharged from the human body, the system comprising:
a body interface device for fitting to the human body for receiving body liquids discharged by the body, the body interface device including a liquid sensor;
a vacuum chamber for storing a vacuum;
a pump coupled to the vacuum chamber for charging the vacuum chamber with a vacuum;
a control valve coupled between the body interface device and the vacuum chamber for controlling the application of aspiration suction from the vacuum chamber to the body interface device; and
a control circuit responsive to the output of the liquid sensor and coupled to control the valve.

21. The aspiration system of claim 20, wherein the control circuit is configured to control the control valve to apply high aspiration suction when the presence of discharged body liquid is detected by the liquid sensor.

22. The aspiration system of claim 20, wherein the control circuit is configured to control the control valve to reduce or stop aspiration suction when the presence of discharge body liquid is not detected by the liquid sensor.

23. The aspiration system of claim 22, wherein the control circuit is configured to control the control valve to continue high aspiration suction for a predetermined time duration once the presence of discharged body liquid is no longer detected by the liquid sensor, whereafter the control circuit controls the control valve to reduce or stop aspiration suction.

24. The aspiration system of claim 20, wherein the pump is controlled to charge the vacuum chamber whenever the pressure within the vacuum chamber exceeds a predetermined threshold.

25. The aspiration system of claim 24, further comprising a vacuum sensor coupled to the vacuum chamber for sensing the level of pressure in the vacuum chamber, and wherein the pump is controlled in response to the signal generated by the vacuum sensor.

26. The aspiration system of claim 20, further comprising a connector for releasably coupling to a collection chamber for collecting aspirated liquid, the connector including a connector valve for preventing escape of the vacuum when the collector chamber is disconnected from the connector.

27. The aspiration system of claim 20, wherein the control valve is selected from: a changeover valve for selecting one of a plurality of flow paths; a continuously variable aperture valve; a variable aperture valve having a plurality of fixed aperture sizes; an open/close valve.

28. The aspiration system of claim 27, wherein the control valve is a changeover valve for selecting between a substantially unrestricted high-vacuum path, and a relatively restricted low-vacuum path.

29. The aspiration system of claim 20, wherein the control circuit comprises a controller having an information map containing pre-programmed control responses to sensed input values.

30. An aspiration unit for coupling to a body interface device to form a system for removing body liquids discharged from the human body, the aspiration unit comprising:
an aspiration connector for coupling to an aspiration path of the body interface device for applying suction thereto;
a signal connector for coupling to a liquid sensor of the body interface device for receiving a signal from the liquid sensor;
a vacuum chamber for storing a vacuum;
a pump for charging the vacuum chamber with a vacuum;
a control valve coupled between the vacuum chamber and the aspiration connector; and
a control circuit coupled to the control valve and to the signal connector, for controlling the control valve to apply aspiration suction from the vacuum chamber in response to a signal from the liquid sensor.

31. A method of detecting, in a body interface device of an aspiration system for removing body liquid discharged by the human body, the presence of discharged body liquid arriving in the body interface device, the method comprising monitoring temperature at the body interface device.

32. A method of detecting, in a body interface device of an aspiration system for removing body liquid discharged by the human body, the presence of discharged body liquid arriving at the body interface, the method comprising monitoring the output of a resistance bridge circuit that includes at least one element having a resistance responsive to the presence of liquid and disposed at said body interface device.

33. A method of applying aspiration suction to a body interface device of an aspiration system for removing body liquid discharged by the human body, the method comprising:
pre-charging a vacuum chamber to store a vacuum;
detecting the presence of liquid arriving at the body interface device; and
controlling a valve to apply aspiration suction from the vacuum chamber to the body interface device, in response to said detection of liquid.

34. The method of claim 33, further comprising operating a pump to charge the vacuum chamber whenever the pressure within the vacuum chamber exceeds a threshold.
